(19) United States
(12) Patent Application Publication
(10) Pub. No.: US 2010/0047298 A1
(43) Pub. Date: Feb. 25, 2010

(54) PROCESS FOR TREATING THE SKIN BY APPLYING SILICONE COMPOUNDS

(75) Inventor: Guillaume Cassin, Villebon sur Yvette (FR)

Correspondence Address: Oliff & Berridge, PLC, P.O. Box 320850, Alexandria, VA, 22320-4850, US

(73) Assignee: L'OREAL, Paris (FR)

(21) Appl. No.: 12/519,271

(22) PCT Filed: Dec. 19, 2007
(86) PCT No.: PCT/FR07/52563

§ 371 Date: Sep. 25, 2009

Related U.S. Application Data
(60) Provisional application No. 60/883,171, filed on Jan. 3, 2007.

(30) Foreign Application Priority Data
Dec. 20, 2006 (FR) 06 55727

(51) Int. Cl. A61K 8/18; A61Q 1/12; A61K 31/74; A61K 31/695; A61K 8/02

(52) U.S. Cl. 424/401; 424/600; 424/69; 424/78.03; 514/63

(57) ABSTRACT

The invention relates to a cosmetic treatment method for improving the superficial appearance of the skin, and especially for reducing the visible and/or tactile irregularities of the skin, such as cutaneous microrelief. According to said method, a filling product is applied to the irregularities, said product resulting from a hydrosilylation reaction, in the presence of a catalyst, between two compounds X and Y, at least one of said compounds being a silicone compound, and both compounds being brought into contact with each other in the presence of a catalyst such that the reaction occurs at least partially on the skin.

The present invention relates to a cosmetic treatment process for improving the surface appearance of the skin by applying at least two compounds X and Y that can react together, where appropriate in the presence of a catalyst or a peroxide, at least one of the compounds being silicone-based.

The treatment process according to the invention is more particularly intended for reducing visible and/or tactile skin irregularities, for instance the cutaneous microrelief, or even deep skin wrinkles.

In the course of the aging process, an impairment in the structure and functions of the skin appears. The main clinical signs observed following this impairment are the appearance of wrinkles and fine lines associated with slackening of the skin.

It is known that such slackening can be immediately corrected by applying to the skin a compound capable of making the skin taut via a tensioning effect, this tension allowing the skin to be smoothed out and the wrinkles and fine lines to be immediately reduced, or even to disappear.

Many tensioning agents acting according to the mechanism described above are already known to date for treating the signs of aging of the skin.

However, the efficacy of such tensioning agents is limited over time, and tends to disappear in the course of the day, the tensioning film especially becoming fractured due to the effect of facial expressions.

What is more, these tensioning agents are effective only on the surface wrinkles of the Skin, and are less effective on the deeper wrinkles, for which their action is, in contrast, very short-lived.

There is thus a need for a cosmetic treatment process, and also for cosmetic compositions and products, for reducing visible and/or tactile skin irregularities, for instance the cutaneous microrelief, or even the deeper wrinkles of the skin, appreciably and throughout the day.

Recently, the inventors have found that it is possible to obtain properties of physical filling of skin irregularities, especially those in the form of “hollows”, by exploiting the ability of certain compounds, especially silicone compounds, to interact when they are placed in contact and to constitute after their interaction a polymeric filling agent.

Thus, compounds known as compound X and compound Y, as defined hereinbelow, prove to be capable of polymerizing in situ, at atmospheric pressure and room temperature, and of forming advantageously biocompatible wrinkle-filling agents, that are capable of physically filling skin irregularities, for instance the cutaneous microrelief, or even deeper skin wrinkles, in a long-lasting manner. Such systems are especially described in part in documents WO 01/96450 and GB 2 407 496.

These polymeric filling agents, which can be formed in situ on a support, especially such as a keratin material, also prove to have advantageous cosmetic properties, namely good adhesion and comfort.

The inventors have discovered, unexpectedly, that it is possible to use these polymeric filling agents in a cosmetic treatment process for improving the surface appearance of the skin.

Thus, according to a first of its aspects, one subject of the present invention is a cosmetic treatment process for improving the surface appearance of the skin, and in particular for reducing visible and/or tactile skin irregularities, for instance the cutaneous microrelief, which consists in applying to said irregularities a filling product resulting from a hydrosilylation reaction in the presence of a catalyst, or a condensation reaction, or a crosslinking reaction in the presence of a peroxide, between two compounds X and Y, at least one of which is a silicone compound, said compounds being placed in contact with each other, where appropriate in the presence of a catalyst or a peroxide, such that said reaction takes place at least partly on the skin.

According to one embodiment of the invention, the filling product may be obtained by extemporaneously mixing a first composition comprising, in a physiologically acceptable medium, at least the compound x, and a second composition comprising, in a physiologically acceptable medium, at least the compound Y, with at least one of said first and second compositions also containing, where appropriate, at least one catalyst or one peroxide.

According to this embodiment, the first and second compositions may advantageously be in the form of an emulsion, or in the form of a paste, which may be anhydrous.

It should be noted that the first and second compositions are different than each other. In one embodiment of the invention, the first composition may be advantageously free of compounds Y and the second composition may be advantageously free of compounds x. Specifically, with regard to their high reactivity toward each other, compounds X and Y are not simultaneously present in a first and/or second composition according to the invention, when their interaction is not conditioned by the presence of a catalyst or a peroxide.

When their interaction is conditioned by the presence of a catalyst or a peroxide, compounds X and Y may be simultaneously present in a composition, if this composition does not comprise any catalyst or peroxide.

Where appropriate, one of the compounds X or Y may be present in a composition simultaneously with a catalyst or a peroxide, if the other compound is not present in this composition.

It is also possible to apply to the skin at least one coat of a first composition comprising, in a physiologically acceptable medium, compound X, and then to deposit on the coat(s) of said first composition at least one coat of a second composition comprising, in a physiologically acceptable medium, compound Y, at least one of said first and second compositions also containing, where appropriate, at least one catalyst or one peroxide.

Thus, according to another of its aspects, a subject of the present invention is also a cosmetic treatment process for improving the surface aspect of the skin, and in particular for reducing visible and/or tactile skin irregularities, for instance the cutaneous microrelief, comprising at least the application to skin presenting said irregularities of a filling product resulting from the application of at least:
one coat of a first composition comprising, in a physiologically acceptable medium, at least one compound X, and
one coat of a second composition comprising, in a physiologically acceptable medium, at least one compound Y,
   at least one of the compounds X and Y being a silicone compound, said compounds X and Y being capable of reacting together via a hydrosilylation reaction in the presence of a catalyst, or via a condensation reaction, or via a crosslinking reaction in the presence of a peroxide, when they are placed in contact with each other, with at least one of said first and second compositions also containing, where appropriate, at least one catalyst or one peroxide.

According to one variant, the process consists in applying to the skin at least one coat of the second composition comprising compound Y, and then in depositing on the coat(s) of said second composition at least one coat of the first composition comprising compound X, at least one of said first and second compositions also comprising, where appropriate, at least one catalyst or one peroxide.

Several coats of each of the first and second compositions may also be applied alternately to skin presenting said irregularities.

According to one embodiment, the cosmetic treatment process according to the invention may also comprise the application of interference particles to visible and/or tactile skin irregularities.

The joint application of interference particles, present within one and/or the other of the first and second compositions, advantageously affords, in addition to the effect resulting from the filling product described previously, an optical effect that in parallel affects the visual perception to the naked eye of the skin irregularities. The concealing effect sought by the process according to the invention is thus reinforced.

According to yet another of its aspects, the invention also relates to a kit for treating keratin materials, especially the skin, comprising at least two different compositions conditioned separately, the kit comprising at least one filler and/or one wax, at least one compound X, at least one compound Y, with at least one of the compounds X or Y being silicone-based, and optionally at least one catalyst or one peroxide, said compounds X and Y being capable of reacting together via a hydrosilylation reaction in the presence of a catalyst, or via a condensation reaction, or via a crosslinking reaction in the presence of a peroxide, when they are placed in contact with each other and in which compounds X and Y, and the catalyst or the peroxide when they are present, are not simultaneously present in the same composition, and said filler and/or wax is in an amount sufficient to impart opacity to the filling product resulting from the interaction of compounds X and Y, after mixing together the compositions of the kit.

In particular, said kit may comprise at least:
one first composition comprising, in a physiologically acceptable medium, at least one compound X,
one second composition comprising, in a physiologically acceptable medium, at least one compound Y,
   at least one of the compounds X and Y being a silicone compound, said compounds X and Y being capable of reacting together via a hydrosilylation reaction in the presence of a catalyst, or via a condensation reaction, or via a crosslinking reaction in the presence of a peroxide, when they are placed in contact with each other, with at least one of the first and second compositions also comprising at least one filler and/or one wax and at least one of said compositions also containing, where appropriate, at least one catalyst or one peroxide.

For the purposes of the invention, it is understood that the mixture formed by mixing such a first composition and such a second composition comprises compounds X and/or Y in a form that has not yet reacted and not exclusively in the form of their product of reaction via hydrosilylation in the presence of a catalyst, via condensation and/or via crosslinking in the presence of a peroxide.

Thus, the formation of the reaction product according to the invention may be performed either directly on the surface of the keratin material that is to be treated, or initiated just before application by extemporaneous mixing of compounds X and Y under conditions that are favorable for their interaction, the formation of the reaction product being, in the latter case, finalized at the surface of the keratin material.

For obvious reasons, and with regard to the high reactivity of compounds X and/or Y, it is in point of fact necessary for their use to be performed under conditions that are favorable to the handleability of the composition containing it (or them), especially for the purpose of spreading it (them), for example. The process in accordance with the invention thus uses a composition containing compounds X and Y, which is thus not set in the form of the expected final film resulting from the reaction of all of X and/or of all of Y.

According to one particular embodiment of the invention, the two compositions have a semi-liquid to pasty texture, and in particular have, for a shear rate equal to 10⁻³ s⁻¹, a viscosity of greater than 10 000 Pa·s and preferably greater than 20 000 Pa·s.

According to another embodiment, at least one of the compositions used in a process according to the invention is anhydrous, and preferably both compositions are anhydrous.

According to yet another particular embodiment of the invention, both compositions or each composition of the kit contain(s) at least one filler and/or one wax in an amount that is sufficient to impart opacity to each of the compositions.

Preferably, the first composition comprising compound X and the second composition comprising the silicone compound Y are conditioned in separate conditioning.

For example, each composition may be conditioned separately in the same conditioning article, for example in a two-compartment pen, the base composition being delivered from one end of the pen and the top composition being delivered from the other end of the pen, each end being closed especially in a leaktight manner by means of a lid. Each composition may also be conditioned in a compartment within the same conditioning article, the mixing of the two compositions taking place at the end(s) of the conditioning article during the delivery of each composition.

Alternatively, each of the first and second compositions may be conditioned in a different conditioning article.

The present invention is also directed toward a cosmetic composition for treating keratin materials, comprising, in a physiologically acceptable medium, at least one compound X, one compound Y, at least one filler and/or wax, said filler and/or wax being present in an amount that is sufficient to impart opacity to the composition, and, where appropriate, at least one catalyst or one peroxide, with at least one of the compounds X or Y being present in an encapsulated form.

According to one particular embodiment of the invention, the composition has a semi-liquid to pasty texture, and may have, for a shear rate equal to 10⁻³ s⁻¹, a viscosity of greater than 10 000 Pa·s and preferably greater than 20 000 Pa·s.

According to a first variant, the applied composition contains at least one of the compounds X and Y in an encapsulated form.

According to one preferred embodiment variant, the two compounds X and Y are present in separate encapsulated forms.

According to this embodiment, the two compounds X and Y may be conditioned in the same composition, where appropriate in the presence of a catalyst or a peroxide, while at the same time overcoming the risk of premature reaction with each other. This reaction takes place only at the moment when the composition is manipulated beforehand or at the time of its application to the skin. The encapsulated form(s) of compound X and/or Y break on drying and compounds X and Y which then come into contact, where appropriate in the presence of a catalyst or a peroxide, react to form the expected filling agent.

Finally, the invention relates to the use of a kit or a cosmetic composition as defined above for obtaining a film deposited on the skin that durably dissimulates skin irregularities, in particular deep facial wrinkles.

The process according to the invention advantageously leads to a deposit that is comfortable on the skin, which durably dissimulates visible and/or tactile skin irregularities.

It is also effective for treating wrinkles and fine lines, and most particularly deep skin wrinkles.

The products and compositions according to the invention have a viscosity that is suitable for the desired use, and especially improved deformability, elasticity and handleability when compared with the products known hitherto.

Thus, the products and cosmetic compositions according to the invention are suitable for modeling the skin, especially facial skin, and enable a reduction in the cutaneous microrelief.

For the purposes of the invention, the expression “visible and/or tactile skin irregularities” is intended to denote the signs of aging of the skin, for instance wrinkles, especially deep wrinkles, and fine lines, and also acne marks, chicken pox marks and scars.

The term “deep wrinkles” is intended to denote the wrinkles caused by aging of the skin, as opposed to “expression wrinkles” that are induced especially by dermal contraction of the facial muscles. It is the tensions responsible for the expression wrinkles that, via pulling on the deep side of the dermis, have a tendency to create hollows and to form deep wrinkles over time.

The present invention is more particularly devoted to the cosmetic treatment of wrinkles, in particular deep skin wrinkles, especially deep wrinkles of facial skin.

The term “semi-liquid to pasty texture” denotes a texture having a viscosity that is sufficient to permit use in the process according to the invention, preferably having, for a shear rate equal to 103 s⁻¹, a viscosity of greater than 10 000 Pa·s and preferably greater than 20 000 Pa·s.

### Measurement of the Opacity

The filling products or the composition as described above may be characterized by an opacity of greater than 35% and preferably greater than 50%.

The composition or the mixture of compositions that is to form the filling product is spread onto a transparent film (Hp Color laser jet transparency, Hp Invent; CP2936A) using an automatic applicator from Braive Instruments (wet thickness of 100 μm). The spreadings are then placed in a thermostatically maintained and ventilated oven for 24 hours at 37° C.

Once dried, these films are placed for evaluation on a contrast card (Prufkarte type 24/5-250 cm² sold by the company Erichsen). The opacity is then measured using a Minolta CR-400 colorimeter from the values Y of the black and white parts obtained in the tristimulus system (X, Y, Z). The opacity value is obtained from the following equation;

(Y black area/Y white area)*100=% opacity

If the film is totally transparent, the opacity is equal to 0.

### Measurement of the Viscosity

As stated previously, the cosmetic compositions according to the invention have a semi-liquid to pasty texture, having, for a shear rate equal to 10⁻³ s⁻¹, a viscosity of greater than 10 000 Pa·s and preferably greater than 20 000 Pa·s, and especially less than 1 000 000 Pa·s.

For their part, the cosmetic kits according to the invention comprise at least one first and one second composition, at least one of the first and second compositions having a semi-liquid to pasty texture, having, for a shear rate equal to 10⁻³ s⁻¹, a viscosity of greater than 10 000 Pa·s and preferably greater than 20 000 Pa·s, and especially less than 1 000 000 Pa·s.

According to one preferred mode of the invention, each of the compositions of the product, for example each of the two compositions of the product, has a semi-liquid to pasty texture, having, for a shear rate equal to 10⁻³ s⁻¹, a viscosity of greater than 10 000 Pa·s and preferably greater than 20 000 Pa·s, and especially less than 1 000 000 Pa·s.

The viscosity of a composition at different shear rates, and in particular at a shear rate equal to 10⁻³ s⁻¹, may be measured especially using a controlled-stress rheometer, for instance a Haake RS150 rheometer, at a temperature of 25° C.

### Compounds X and Y

The term “silicone compound” means a polyorganosiloxanes compound, i.e. a compound comprising at least two organosiloxane units, for example at least 5 organosiloxane units and especially at least 10 organosiloxane units. According to one particular embodiment, at least one of the compounds X and Y, or the compounds X and the compounds Y, is (are) silicones based. Compounds X and Y may be aminated or non-aminated.

According to another embodiment, at least one of the compounds X and Y is a polymer whose main chain is predominantly formed from organosiloxane units. Among the silicone compounds mentioned below, some may simultaneously have film-forming properties and adhesive properties, depending, for example, on the silicone proportion thereof or on whether they are used as a mixture with a particular additive. It is consequently possible to modify the film-forming properties or the adhesive properties of such compounds according to the intended use, and this is in particular the case for reactive silicone elastomers known as “room-temperature-vulcanizable” elastomers.

Compounds X and Y may react together at a temperature ranging between room temperature and 180° C. Advantageously, compounds X and Y can react together at room temperature (20±5° C.) and atmospheric pressure, or advantageously in the presence of a catalyst, via a hydrosilylation reaction or a condensation reaction, or a crosslinking reaction in the presence of a peroxide.

### Polar Groups

According to one particular embodiment, at least one of the compounds X and Y, for example compound X, bears at least one polar group capable of forming at least one hydrogen bond with keratin materials.

The term “polar group” means a group comprising carbon and hydrogen atoms in its chemical structure and at least one heteroatom (such as O, N, S and P), such that said group is capable of establishing at least one hydrogen bond with keratin materials.

Compounds bearing at least one group capable of establishing a hydrogen bond are particularly advantageous, since they afford the compositions containing them better adhesion to keratin materials.

The polar group(s) borne by at least one of the compounds X and Y is (are) capable of establishing a hydrogen bond, and comprise(s) either a hydrogen atom bonded to an electronegative atom, or an electronegative atom, for instance an oxygen, nitrogen or sulfur atom. When the group comprises a hydrogen atom bonded to an electronegative atom, the hydrogen atom may interact with another electronegative atom borne, for example, by another molecule, such as keratin, to form a hydrogen bond. When the group comprises an electronegative atom, the electronegative atom may interact with a hydrogen atom bonded to an electronegative atom borne, for example, by another molecule, such as keratin, to form a hydrogen bond.

Advantageously, these polar groups may be chosen from the following groups:
carboxylic acid —COOH,
alcohols, such as: —CH₂OH or —CH(R)OH, R being an alkyl radical containing from 1 to 6 carbon atoms,
amino of formula —NR₁R₂, in which R₁ and R₂, which may be identical or different, represent an alkyl radical containing from 1 to 6 carbon atoms, or R₁ or R₂ denotes a hydrogen atom, and the other from among R₁ and R₂ represents an alkyl radical containing from 1 to 6 carbon atoms,
pyridino,
amido of formula —NH—COR′ or —CO—NH—R′ in which R′ represents a hydrogen atom or an alkyl radical containing from 1 to 6 carbon atoms,
pyrrolidino preferably chosen from the groups of formula:

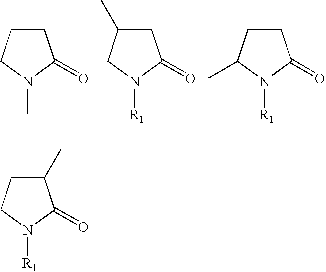

R₁ being an alkyl radical containing from 1 to 6 carbon atoms,
carbamoyl of formula —O—CO—NH—R′ or —NH—CO—OR′, R′ being as defined above,
thiocarbamoyl such as —O—CS—NH—R′ or —NH—CS—OR′, R′ being as defined above,
ureyl such as —NR′—CO—N(R′)₂, the groups R′, which may be identical or different, being as defined above,
sulfonamido such as NR′—S(═O)₂—R′, R′ corresponding to the above definition.

Preferably, these polar groups are present in a content of less than or equal to 10% by weight, preferably less than or equal to 5% by weight, for example in a content ranging from 1% to 3% by weight, relative to the weight of each compound X or Y.

The polar group(s) may be located in the main chain of compound X and/or Y or may be pendent on the main chain or located at the ends of the main chain of compound X and/or Y.

### 1—Compounds X and Y Capable of Reacting Via Hydro-Silylation

According to one embodiment, the invention relates to a cosmetic treatment process for improving the surface appearance of the skin, and in particular for reducing visible and/or tactile skin irregularities, for instance the cutaneous microrelief, which consists in applying to said irregularities a filling product resulting from a hydrosilylation reaction in the presence of a catalyst between two compounds X and Y, at least one of which is a silicone compound, said compounds being placed in contact with each other in the presence of a catalyst, such that said reaction takes place at least partly on the skin.

According to this embodiment, compounds X and Y are capable of reacting via hydrosilylation in the presence of a catalyst, this reaction being able to be represented schematically in simple terms as follows:

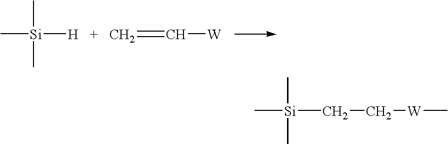

with W representing a carbon-based and/or silicone chain containing one or more unsaturated aliphatic groups.

In this case, compound X may be chosen from silicone compounds comprising at least two unsaturated aliphatic groups. For example, compound X may comprise a silicone main chain whose unsaturated aliphatic groups are pendent on the main chain (side group) or located at the ends of the main chain of the compound (end group). In the rest of the description, these particular compounds will be referred to as polyorganosiloxanes containing unsaturated aliphatic groups.

According to one embodiment, compound X and/or compound Y bears at least one polar group, as described above, capable of forming at least one hydrogen bond with keratin materials. This polar group is advantageously borne by compound X that comprises at least two unsaturated aliphatic groups.

According to one embodiment, compound X is chosen from polyorganosiloxanes comprising at least two unsaturated aliphatic groups, for example two or three vinyl or allylic groups, each bonded to a silicon atom.

According to one advantageous embodiment, compound X is chosen from polyorganosiloxanes comprising siloxane units of formula:

$${R}_{m} {R}^{′} {SiO}_{\frac{( 3 - m )}{2}} \qquad ( I )$$

in which:
R represents a linear or cyclic monovalent hydro-carbon-based group containing from 1 to 30 carbon atoms, preferably from 1 to 20 and better still from 1 to 10 carbon atoms, for instance a short-chain alkyl radical containing, for example, from 1 to 10 carbon atoms, in particular a methyl radical, or alternatively a phenyl group, preferably a methyl radical,
m is equal to 1 or 2, and
R′ represents:
   an unsaturated aliphatic hydrocarbon-based group containing from 2 to 10 and preferably from 3 to 5 carbon atoms, for instance a vinyl group or a group —R″—CH═CHR′″ in which R″ is a divalent aliphatic hydrocarbon-based chain containing from 1 to 8 carbon atoms, bonded to the silicon atom and R′″ is a hydrogen atom or an alkyl radical containing from 1 to 4 carbon atoms, preferably a hydrogen atom; groups R′ that may be mentioned include vinyl and allylic groups and mixtures thereof; or
   an unsaturated cyclic hydrocarbon-based group containing from 5 to 8 carbon atoms, for instance a cyclohexenyl group.

Preferably, R′ is an unsaturated aliphatic hydrocarbon-based group, preferably a vinyl group.

According to one embodiment, R represents an alkyl radical containing from 1 to 10 carbon atoms or a phenyl group, and preferably a methyl radical, and R′is a vinyl group.

According to one particular embodiment, the polyorgano-siloxane also comprises units of formula:

$${R}_{n} {SiO}_{\frac{( 4 - n )}{2}} \qquad ( II )$$

in which R is a group as defined above, and n is equal to 1, 2 or 3.

According to one variant, compound X may be a silicone resin comprising at least two ethylenic unsaturations, said resin being capable of reacting with compound Y via hydrosilylation in the presence of a catalyst. Examples that may be mentioned include resins of MQ or MT type themselves bearing —CH═CH₂ unsaturated reactive ends.

These resins are crosslinked organosiloxane polymers.

The nomenclature of silicone resins is known under the name “MDTQ”, the resin being described as a function of the various siloxane monomer units it comprises, each of the letters M, D, T and Q characterizing a type of unit.

The letter M represents the monofunctional unit of formula (CH₃)₃SiO_{1/2}, the silicon atom being bonded to only one oxygen atom in the polymer comprising this unit.

The letter D means a difunctional unit (CH₃)₂SiO_{2/2} in which the silicon atom is bonded to two oxygen atoms.

The letter T represents a trifunctional unit of formula (CH₃) SiO_{3/2}.

In the units M, D and T defined above, at least one of the methyl groups may be substituted with a group R other than a methyl group, such as a hydrocarbon-based radical (especially alkyl) containing from 2 to 10 carbon atoms or a phenyl group, or alternatively a hydroxyl group.

Finally, the Letter Q means a tetrafunctional unit SiO_{4/2} in which the silicon atom is bonded to four hydrogen atoms, which are themselves bonded to the rest of the polymer. Examples of such resins that may be mentioned include MT silicone resins such as poly(phenylvinylsilsesquioxane), for instance the product sold under the reference SST-3PV1 by the company Gelest.

Preferably, compounds X comprise from 0.01% to 1% by weight of unsaturated aliphatic groups.

Advantageously, compound X is chosen from polyorgano-polysiloxanes, especially those comprising the siloxane units (I) and optionally (II) described above.

Compound Y preferably comprises at least two free Si—H groups (hydrogenosilane groups).

Compound Y may be chosen advantageously from polyorganosiloxanes comprising at least one alkylhydrogenosiloxane unit having the following formula:

$${R}_{p} {HSiO}_{\frac{( 3 - p )}{2}} \qquad ( III )$$

in which:

R represents a linear or cyclic monovalent hydrocarbon-based group containing from 1 to 30 carbon atoms, for instance an alkyl radical containing from 1 to 30 carbon atoms, preferably from 1 to 20 and better still from 1 to 10 carbon atoms, in particular a methyl radical, or alternatively a phenyl group, and p is equal to 1 or 2. Preferably, R is a hydrocarbon-based group, preferably methyl.

These polyorganosiloxane compounds Y containing alkyl-hydrogenosiloxane units may also comprise units of formula:

$${R}_{n} {SiO}_{\frac{( 4 - n )}{2}} \qquad ( II )$$

as defined above.

Compound Y may be a silicone resin comprising at least one unit chosen from the units M, D, T and Q as defined above and comprising at least one Si—H group, such as the poly(methylhydridosilsesquioxanes) sold under the reference SST-3 MH1.1 by the company Gelest.

Preferably, these polyorganosiloxane compounds y comprise from 0.5% to 2.5% by weight of Si—H groups.

Advantageously, the radicals R represent a methyl group in formulae (I), (II) and (III) above.

Preferably, these polyorganosiloxanes Y comprise end groups of formula (CH₃)₃SiO_{1/2}.

Advantageously, the polyorganosiloxanes Y comprise at least two alkylhydrogenosiloxane units of formula (H₃C) (H)SiO and optionally comprise (H₃C)₂SiO units.

Such polyorganosiloxane compounds Y containing hydrogenosilane groups are described, for example, in document EP 0 465 744.

According to one variant, compound X is chosen from organic oligomers or polymers (the term “organic” means compounds whose main chain is not silicone-based, preferably compounds comprising no silicon atoms) or from organic/silicone hybrid polymers or oligomers, said oligomers or polymers bearing at least 2 reactive unsaturated aliphatic groups, compound Y being chosen from the polyorganosiloxanes Y containing hydrogeno-siloxane groups mentioned above.

According to one embodiment, the organic or hybrid organic/silicone compounds X bearing at least 2 reactive unsaturated aliphatic groups bear at least one polar group as described above.

Compound X, of organic nature, may then be chosen from vinyl or (meth)acrylic polymers or oligomers, polyesters, polyurethanes and/or polyureas, polyethers, perfluoropolyethers, polyolefins such as polybutene or polyisobutylene, dendrimers and organic hyperbranched polymers, or mixtures thereof.

In particular, the organic polymer or the organic part of the hybrid polymer may be chosen from the following polymers:

a) ethylenically unsaturated polyesters:

This is a group of polymers of polyester type containing at least 2 ethylenic double bonds, randomly distributed in the main polymer chain. These unsaturated polyesters are obtained by polycondensation of a mixture:
of linear or branched aliphatic or cycloaliphatic dicarboxylic acids especially containing from 3 to 50 carbon atoms, preferably from 3 to 20 and better still from 3 to 10 carbon atoms, such as adipic acid or sebacic acid, of aromatic dicarboxylic acids especially containing from 8 to 50 carbon atoms, preferably from 8 to 20 and better still from 8 to 14 carbon atoms, such as phthalic acids, especially terephthalic acid, and/or of dicarboxylic acids derived from ethylenically unsaturated fatty acid dimers such as the oleic or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 and better still from 6 to 15 carbon atoms, such as bisphenol A and bisphenol B, and/or of diol dimers obtained from the reduction of fatty acid dimers as defined above, and
of one or more dicarboxylic acids or anhydrides thereof comprising at least one polymerizable ethylenic double bond and containing from 3 to 50 carbon atoms, preferably from 3 to 20 and better still from 3 to 10 carbon atoms, such as maleic acid, fumaric acid or itaconic acid.
   b) polyesters containing (meth)acrylate side groups and/or end groups:

This is a group of polymers of polyester type obtained by polycondensation of a mixture:
of linear or branched aliphatic or cycloaliphatic dicarboxylic acids especially containing from 3 to 50 carbon atoms, preferably from 3 to 20 and better still from 3 to 10 carbon atoms, such as adipic acid or sebacic acid, of aromatic dicarboxylic acids especially containing from 8 to 50 carbon atoms, preferably from 8 to 20 and better still from 8 to 14 carbon atoms, such as phthalic acids, especially terephthalic acid, and/or of dicarboxylic acids derived from ethylenically unsaturated fatty acid dimers such as the oleic acid or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 and better still from 6 to 15 carbon atoms, such as bisphenol A and bisphenol B, and
of at least one ester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate or glycerol methacrylate.

These polyesters differ from those described above in point a) by the fact that the ethylenic double bonds are not located in the main chain but on side groups or at the end of the chains. These ethylenic double bonds are those of the (meth)acrylate groups present in the polymer.

Such polyesters are sold, for example, by the company UCB under the names Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50 000: molar mass 1500, on average 6 acrylate functions per molecule).

c) polyurethanes and/or polyureas containing (meth)-acrylate groups, obtained by polycondensation
of aliphatic, cycloaliphatic and/or aromatic diisocyanates, triisocyanates and/or polyisocyanates especially containing from 4 to 50 and preferably from 4 to 30 carbon atoms, such as hexamethylene diisocyanate, isophorone diisocyanate, toluene diisocyanate, diphenylmethane diisocyanate or isocyanurates of formula

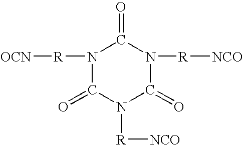

resulting from the trimerization of 3 molecules of diisocyanates OCN—R—CNO, in which R is a linear, branched or cyclic hydrocarbon-based radical comprising from 2 to 30 carbon atoms;
of polyols, especially of diols, free of polymerizable ethylenic unsaturations, such as 1,4-butanediol, ethylene glycol or trimethylolpropane, and/or of aliphatic, cycloaliphatic and/or aromatic polyamines, especially diamines, especially containing from 3 to 50 carbon atoms, such as ethylenediamine or hexamethylenediamine, and
of at least one ester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate or glycerol methacrylate.

Such polyurethanes/polyureas containing acrylate groups are sold, for example, under the name SR 368 (tris(2-hydroxyethyl)isocyanurate-triacrylate) or Craynor® 435 by the company Cray Valley, or under the name Ebecryl® by the company UCB (Ebecryl® 210: molecular mass 1500, 2 acrylate functions per molecule, Ebecryl® 230: molecular mass 5000, 2 acrylate functions per molecule, Ebecryl® 270: molecular mass 1500, 2 acrylate functions per molecule, Ebecryl® 8402: molecular mass 1000, 2 acrylate functions per molecule, Ebecryl® 8804: molecular mass 1300, 2 acrylate functions per molecule, Ebecryl® 220: molecular mass 1000, 6 acrylate functions per molecule, Ebecryl® 2220: molecular mass 1200, 6 acrylate functions per molecule, Ebecryl® 1290: molecular mass 1000, 6 acrylate functions per molecule, Ebecryl® 800: molecular mass 800, 6 acrylate functions per molecule).

Mention may also be made of the water-soluble aliphatic diacrylate polyurethanes sold under the names Ebecryl® 2000, Ebecryl® 2001 and Ebecryl® 2002, and the diacrylate polyurethanes in aqueous dispersion sold under the trade names IRR® 390, IRR® 400, IRR® 422 and IRR® 424 by the company UCB.

d) polyethers containing (meth)acrylate groups obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide preferably having a weight-average molecular mass of less than 10 000, and polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by the company Cray Valley or under the name Ebecryl® 11 by UCB. Polyethoxylated trimethylolpropane triacrylates are sold, for example, under the names SR 454, SR 498, SR 502, SR 9035 and SR 415 by the company Cray Valley or under the name Ebecryl® 160 by the company UCB. Polypropoxylated trimethylolpropane triacrylates are sold, for example, under the names SR 492 and SR 501 by the company Cray Valley.

e) epoxyacrylates obtained by reaction between
at least one diepoxide chosen, for example, from:
(i) bisphenol A diglycidyl ether,
(ii) a diepoxy resin resulting from the reaction between bisphenol A diglycidyl ether and epichlorohydrin,
(iii) an epoxy ester resin containing α,ω-diepoxy end groups resulting from the condensation of a dicarboxylic acid containing from 3 to 50 carbon atoms with a stoichiometric excess of (i) and/or (ii), and
(iv) an epoxy ether resin containing α,ω-diepoxy end groups resulting from the condensation of a diol containing from 3 to 50 carbon atoms with a stoichiometric excess of (i) and/or (no),
(v) natural or synthetic oils bearing at least 2 epoxide groups, such as epoxidized soybean oil, epoxidized linseed oil or epoxidized vernonia oil,
(vi) a phenol-formaldehyde polycondensate (Novolac® resin), the end groups and/or side groups of which have been epoxidized,
   and
one or more carboxylic acids or polycarboxylic acids comprising at least one ethylenic double bond in the α,β-position relative to the carboxylic group, for instance (meth)acrylic acid or crotonic acid or esters of (meth)acrylic acid and of a diol or polyol containing from 2 to 20 carbon atoms and preferably from 2 to 6 carbon atoms, such as 2-hydroxyethyl (meth)acrylate.

Such polymers are sold, for example, under the names SR 349, SR 601, CD 541, SR 602, SR 9036, SR 348, CD 540, SR 480 and CD 9038 by the company Cray Valley, under the names Ebecryl® 600, Ebecryl® 609, Ebecryl® 150, Ebecryl® 860 and Ebecryl® 3702 by the company UCB and under the names Photomer® 3005 and Photomer® 3082 by the company Henkel.

f) poly(C₁₋₅₀ alkyl (meth)acrylates), said alkyl being linear, branched or cyclic, comprising at least two functions containing an ethylenic double bond borne by the hydrocarbon-based side chains and/or end chains.

Such copolymers are sold, for example, under the names IRR® 375, OTA® 480 and Ebecryl® 2047 by the company UCB.

g) polyolefins such as polybutene or polyisobutylene,

h) perfluoropolyethers containing acrylate groups obtained by esterification, for example with (methyacrylic acid, of perfluoropolyethers bearing hydroxyl side groups and/or end groups.

Such α,ω-diol perfluoropolyethers are described especially in EP-A-1 057 849 and are sold by the company Ausimont under the name Fomblin® Z Diol.

i) hyperbranched dendrimers and polymers bearing (meth)acrylate or (meth)acrylamide end groups obtained, respectively, by esterification or amidation of hyperbranched dendrimers and polymers containing hydroxyl or amino end functions, with (meth)acrylic acid.

Dendrimers (from the Greek dendron=tree) are “arborescent”, i.e. highly branched, polymer molecules invented by D. A. Tomalia and his team at the start of the 1990s (Donald A. Tomalia et al., Angewandte Chemie, Int. Engl. Ed., Vol. 29, No. 2, pages 138-175). These are structures constructed about a central unit that is generally polyvalent. About this central unit are linked, in a fully determined structure, branched chain-extending units, thus giving rise to monodispersed symmetrical macromolecules having a well-defined chemical and stereochemical structure. Dendrimers of polyamidoamine type are sold, for example, under the name Starburst® by the company Dendritech.

Hyperbranched polymers are polycondensates, generally of polyester, polyamide or polyethyleneamine type, obtained from multifunctional monomers, which have an arborescent structure similar to that of dendrimers but are much less regular than dendrimers (see, for example, WO-A-93/17060 and Wo 96/12754).

The company Perstorp sells hyperbranched polyesters under the name Boltorn®. Hyperbranched polyethylene-amines will be found under the name Comburst® from the company Dendritech. Hyperbranched poly(esteramides) containing hydroxyl end groups are sold by the company DSM under the name Hybrane®.

These hyperbranched dendrimers and polymers esterified or amidated with acrylic acid and/or methacrylic acid are distinguished from the polymers described in points a) to h) above by the very large number of ethylenic double bonds present. This high functionality, usually greater than 5, makes them particularly useful by allowing them to act as “crosslinking nodes”, i.e. sites of multiple crosslinking.

These dendritic and hyperbranched polymers may thus be used in combination with one or more of the polymers and/or oligomers a) to h) above.

1a—Additional Reactive Compounds

According to one embodiment, the compositions comprising compound X and/or Y may also comprise an additional reactive compound such as:
organic or mineral particles comprising at their surface at least 2 unsaturated aliphatic groups: examples that may be mentioned include silicas surface-treated, for example, with silicone compounds containing vinyl groups, for instance cyclotetramethyl-tetravinylsiloxane-treated silica,
silazane compounds such as hexamethyldisilazane.
   1b—Catalyst

The hydrosilylation reaction is performed in the presence of a catalyst that may be present with one or the other of the compounds X or Y or may be present in isolation. For example, this catalyst may be present in the composition in an encapsulated form if the two compounds X and Y, whose interaction it must bring about, are present in this same composition in an unencapsulated form or, conversely, it may be present therein in an unencapsulated form if at least one of the compounds X and Y is present in the composition in an encapsulated form. The catalyst is preferably platinum-based or tin-based.

Examples that may be mentioned include platinum-based catalysts deposited on a support of silica gel or charcoal powder (coal), platinum chloride, platinum salts and chloroplatinic acids.

Chloroplatinic acids in hexahydrate or anhydrous form, which are readily dispersible in organosilicone media, are preferably used.

Mention may also be made of platinum complexes such as those based on chloroplatinic acid hexahydrate and on divinyltetramethyldisiloxane.

The catalyst may be present in a content ranging from 0.0001% to 20% by weight relative to the total weight of the composition comprising it.

Compounds X and/or Y may be combined with polymerization inhibitors or retardants, and more particularly catalyst inhibitors. Mention may be made, in a nonlimiting manner, of cyclic polymethylvinyl-siloxanes, and in particular tetravinyltetramethyl-cyclotetrasiloxane, and acetylenic alcohols, which are preferably volatile, such as methylisobutynol.

The presence of ionic salts such as sodium acetate may have an influence on the rate of polymerization of the compounds.

By way of example of a combination of compounds X and Y that react via hydrosilylation in the presence of a catalyst, mention may be made of the following references sold by the company Dow Corning: DC 7-9800 Soft Skin Adhesive Parts A & B, and also the combination of mixtures A and B below sold by the company Dow Corning:

| | | | |
|---|---|---|---|
| Ingredient (INCI name) | CAS No. | Content (%) | Function |

MIXTURE A:

| | | | |
|---|---|---|---|
| Dimethylsiloxane, | 68083-19-2 | 55-95 | Polymer |
| Dimethylvinylsiloxy- | | | |
| terminated | | | |
| Silica Silylate | 68909-20-6 | 10-40 | Filler |
| 1,3-Diethenyl-1,1,3,3-Tetra- | 68478-92-2 | Trace | Catalyst |
| methyldisiloxane complexes | | | |
| Tetramethyldivinyldisiloxane | 2627-95-4 | 0.1-1 | Polymer |

MIXTURE B:

| | | | |
|---|---|---|---|
| Dimethylsiloxane, | 68083-19-2 | 55-95 | Polymer |
| Dimethylvinylsiloxy- | | | |
| terminated | | | |
| Silica Silylate | 68909-20-6 | 10-40 | Filler |
| Dimethyl, | 68037-59-2 | 1-10 | Polymer |
| Methylhydrogenosiloxane, | | | |
| trimethylsiloxy-terminated | | | |

Advantageously, compounds X and Y are chosen from silicone compounds capable of reacting via hydrosilylation in the presence of a catalyst; in particular, compound X is chosen from polyorganosiloxanes comprising units of formula (I) described above and compound Y is chosen from organosiloxanes comprising alkylhydrogenosiloxane units of formula (III) described above.

According to one particular embodiment, compound X is a polydimethylsiloxane containing vinyl end groups and compound Y is a polymethylhydrogenosiloxane.

### 2—Compounds X and Y Capable of Reacting Via Condensation

According to one embodiment, the invention relates to a cosmetic treatment process for improving the surface appearance of the skin, and in particular for reducing visible and/or tactile skin irregularities, for instance the cutaneous microrelief, which consists in applying to said irregularities a filling product resulting from a condensation reaction, optionally in the presence of a catalyst, between two compounds X and Y, at least one of which is a silicone compound, said compounds being placed in contact with each other, optionally in the presence of a catalyst, such that said reaction takes place at least partly on the skin.

According to this embodiment, compounds X and Y are capable of reacting via condensation, either in the presence of water (hydrolysis) by reaction of 2 compounds bearing alkoxysilane groups, or via “direct” condensation by reaction of a compound bearing alkoxy-silane group(s) and a compound bearing silanol group(s) or by reaction of 2 compounds bearing silanol group(s).

When the condensation is performed in the presence of water, this water may in particular be ambient moisture, residual water of the skin, the lips, the eyelashes and/or the nails, or the water provided by an external source, for example premoistening of the keratin fibers (for example with a mister, or natural or artificial tears).

In this mode of reaction via condensation, compounds X and Y, which may be identical or different, may thus be chosen from silicone compounds whose main chain comprises at least two alkoxysilane groups and/or at least two silanol (Si—OH) groups, on the side and/or at the end of the chain.

According to one embodiment, compound X and/or compound Y bears at least one polar group, as described above, capable of forming at least one hydrogen bond with keratin materials.

According to one advantageous embodiment, compounds X and/or Y are chosen from polyorganosiloxanes comprising at least two alkoxysilane groups. The term “alkoxysilane group” means a group comprising at least one —Si—OR portion, R being an alkyl group containing from 1 to 6 carbon atoms.

Compounds X and Y are especially chosen from poly-organosiloxanes comprising alkoxysilane end groups, more specifically those comprising at least 2 alkoxysilane end groups and preferably trialkoxysilane end groups.

These compounds X and/or Y, which may be identical or different, preferably predominantly comprise units of formula:

R⁹ₛSiO_{(4-s)/2}, (IV)

in which R⁹ independently represents a radical chosen from alkyl groups containing from 1 to 6 carbon atoms, phenyl and fluoroalkyl groups, and s is equal to 0, 1, 2 or 3. Preferably, R⁹ independently represents an alkyl group containing from 1 to 6 carbon atoms. Alkyl groups that may especially be mentioned include methyl, propyl, butyl, and hexyl, and mixtures thereof, preferably methyl or ethyl. A fluoroalkyl group that may be mentioned is 3,3,3-trifluoropropyl.

According to one particular embodiment, compounds X and Y, which may be identical or different, are polyorgano-siloxanes comprising units of formula:

(R⁹₂SiO₂)_{f}— (V)

in which 9 is as described above, preferably R⁹ is a methyl radical, and f is such that the polymer advantageously has a viscosity at 25° C. ranging from 0.5 to 3000 Pa·s and preferably ranging from 5 to 150 Pa·s. f is especially a number ranging from 2 to 5000, particularly from 3 to 3000 and more particularly from 5 to 1000.

These polyorganosiloxane compounds X and Y comprise at least 2 trialkoxysilane end groups per polymer molecule, said groups having the following formula:

—ZSiR¹ₓ(OR)₃₋ₓ, (VI)

in which:
the radicals R independently represent a methyl, ethyl, n-propyl, isopropyl, n-butyl, sec-butyl or isobutyl group, preferably a methyl or ethyl group,
R¹ is a methyl or ethyl group,
x is equal to 0 or 1 and preferably x is equal to 0, and
Z is chosen from: divalent hydrocarbon-based groups not comprising any ethylenic unsaturation and containing from 1 to 18 preferably from 2 to 18 carbon atoms (alkylene groups), combinations of divalent hydrocarbon-based radicals and of siloxane segments of formula (IX) below:

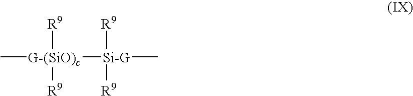

R⁹ being as described above, G is a divalent hydrocarbon-based radical not comprising any ethylenic unsaturation and containing from 1 to 18 carbon atoms and preferably from 2 to 18 carbon atoms, and c is an integer ranging from 1 to 6.

Z and G may be chosen especially from alkylene groups such as ethylene, propylene, butylene, pentylene and hexylene, and arylene groups such as phenylene.

Preferably, Z is an alkylene group, and better still ethylene.

These polymers may contain on average at least 1.2 tri-alkoxysilane end groups or end chains per molecule, and preferably on average at least 1.5 trialkoxysilane end groups per molecule. Since these polymers may contain at least 1.2 trialkoxysilane end groups per molecule, some may comprise other types of end groups such as end groups of formula CH₂—CH═SiR⁹₂— or of formula R⁶₃—Si—, in which R⁹ is as defined above and each group R⁶ is independently chosen from groups R⁹ and vinyl. Examples of such end groups that may be mentioned include trimethoxysilane, triethoxysilane, vinyldimethoxysilane and vinylmethyloxyphenylsilane groups.

Such polymers are especially described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the content of which is incorporated into the present patent application by reference.

As compound X and/or Y, mention may be made in particular of polyorganosiloxanes chosen from the polymers of formula:

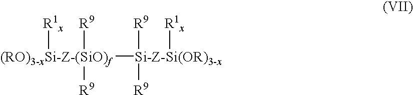

in which R, R¹, R⁹, Z, xx and f are as described above.

Compounds X and/or Y may also comprise a mixture of polymer of formula (VII) above with polymers of formula (VIII) below:

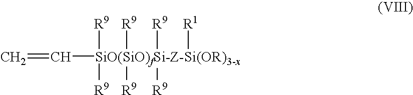

in which R, R¹, R⁹, Z, x and f are as described above.

When the polyorganosiloxane compound X and/or Y containing alkoxysilane group(s) comprises such a mixture, the various polyorganosiloxanes are present in contents such that the organosilyl end chains represent less than 40% and preferably less than 25% by number of the end chains.

The polyorganosiloxane compounds X and/or Y that are particularly preferred are those of formula (VII) described above. Such compounds X and/or Y are described, for example, in document WO 01/96450.

As indicated above, compounds X and Y may be identical or different.

In particular, compounds X and Y may represent a mixture of polydimethylsiloxanes containing methoxy-silane groups.

According to one variant, one of the two reactive compounds X or Y is of silicone nature and the other is of organic nature. For example, compound X is chosen from organic oligomers or polymers or organic/silicone hybrid oligomers or polymers, said polymers or oligomers comprising at least two alkoxysilane groups, and Y is chosen from silicone compounds such as the polyorganosiloxanes described above. In particular, the organic oligomers or polymers are chosen from vinyl, (meth)acrylic, polyester, polyamide, polyurethane and/or polyurea, polyether, polyolefin or perfluoro-polyether oligomers or polymers, and hyperbranched organic dendrimers and polymers, and mixtures thereof.

According to one embodiment, compound X of organic nature or of hybrid organic/silicone nature bears at least one polar group, as described above, capable of forming at least one hydrogen bond with the keratin material.

The organic polymers of vinyl or (meth)acrylic nature bearing alkoxysilane side groups may in particular be obtained via copolymerization of at least one organic vinyl or (meth)acrylic monomer with a (meth)acryloxy-propyltrimethoxysilane, a vinyltrimethoxysilane, a vinyltriethoxysilane, an allyltrimethoxysilane, etc.

Examples that may be mentioned include the (meth)acrylic polymers described in the document by Kusabe, M., Pitture e Verniei—European Coating; 12-B, pages 43-49, 2005, and especially the polyacrylates containing alkoxysilane groups referenced as MAX from Kaneka or those described in the publication by Probster, M., Adhesion-Kleben & Dichten, 2004, 481 (1-2), pages 12-14.

The organic polymers resulting from a polycondensation or a polyaddition, such as polyesters, polyamides, polyurethanes and/or polyureas, and polyethers, and bearing alkoxysilane side and/or end groups, may result, for example, from the reaction of an oligomeric prepolymer as described above with one of the following silane coreagents bearing at least one alkoxysilane group: aminopropyltrimethoxysilane, aminopropyltri-ethoxysilane, aminoethylaminopropyltrimethoxysilane, glycidoxypropyltrimethoxysilane, glycidoxypropyltri-ethoxysilane, epoxycyclohexylethyltrimethoxysilane, mercaptopropyltrimethoxysilane.

Examples of polyethers and polyisobutylenes containing alkoxysilane groups are described in the publication by Kusabe, M., Pitture e Verniei—European Coating; 12-B, pages 43-49, 2005. As examples of polyurethanes containing alkoxysilane end groups, mention may be made of those described in the document by Probster, M., Adhesion-Kleben & Dichten, 2004, 481 (1-2) pages 12-14 or those described in the document by Landon, S., Pitture e Verniei vol. 73, No. 11, pages 18-24, 1997 or in the document by Huang, Mowo, Pitture e vernier vol. 5, 2000, pages 61-67; mention may be made especially of the polyurethanes containing alkoxysilane groups from OSI-WITCO-GE.

Polyorganosiloxane compounds X and/or Y that may be mentioned include resins of MQ or MT type themselves bearing alkoxysilane and/or silanol ends, for instance the poly(isobutylsilsesquioxane) resins functionalized with silanol groups sold under the reference SST-S7C41 (3 Si—OH groups) by the company Gelest.

2a—Additional Reactive Compound

According to one embodiment, compound X and/or Y may also be combined with an additional reactive compound comprising at least two alkoxysilane or silanol groups.

Mention may be made, for example, of:
one or more organic or mineral particles comprising at their surface alkoxysilane and/or silanol groups, for instance fillers surface-treated with such groups.
   2b—Catalyst

The condensation reaction may be performed in the presence of a metal-based catalyst that may be present in one or the other of the compounds X or Y or may be present in isolation. For example, this catalyst may be present in the composition in an encapsulated form if the two compounds X and Y, whose interaction it must bring about, are present in this same composition in an unencapsulated form or, conversely, it may be present in an unencapsulated form if at least one of the compounds X and Y is present in the composition in an encapsulated form. The catalyst that is useful in this type of reaction is preferably a titanium-based catalyst.

Mention may be made especially of the tetraalkoxy-titanium-based catalysts of formula

Ti(OR²)_{y}(OR³)_{4-y},

in which R² is chosen from tertiary alkyl radicals such as tert-butyl, tert-amyl and 2,4-dimethyl-3-pentyl; R³ represents an alkyl radical containing from 1 to 6 carbon atoms, preferably a methyl, ethyl, n-propyl, isopropyl, n-butyl, sec-butyl or hexyl group and y is a number ranging from 3 to 4 and better still from 3.4 to 4.

The catalyst may be present in a content ranging from 0.0001% to 20% by weight relative to the total weight of the composition containing it.

2c—Diluent

The compositions that are useful comprising X and/or Y may also comprise a volatile silicone oil (or diluent) for reducing the viscosity of the composition. This oil may be chosen from short-chain linear silicones such as hexamethyldisiloxane or octamethyltrisiloxane, and cyclic silicones such as octamethylcyclotetrasiloxane and decamethylcyclopentasiloxane, and mixtures thereof.

This silicone oil may represent from 5% to 95% and preferably from 10% to 80% by weight relative to the weight of each composition.

As examples of a combination of compounds X and Y bearing alkoxysilane groups and reacting via condensation, mention may be made of the mixtures A′ and B′ below sold by the company Dow Corning.

| | | | |
|---|---|---|---|
| Ingredient (INCI name) | CAS No. | Content (%) | Function |

Mixture A′:

| | | | |
|---|---|---|---|
| Bis-Trimethoxysiloxyethyl | PMN87176 | 25-45 | Polymer |
| Tetramethyldisiloxyethyl | | | |
| Dimethicone (1) | | | |
| Silica Silylate | 68909-20-6 | 5-20 | Filler |
| Disiloxane | 107-46-0 | 30-70 | Solvent |

Mixture B′:

| | | | |
|---|---|---|---|
| Disiloxane | 107-46-0 | 80-99 | Solvent |
| Tetra-t-butyl Titanate | — | 1-20 | Catalyst |

it should be noted that the identical compounds X and Y are combined in the mixture A′ (cf. (1)).

### 3—Crosslinking in the Presence of Peroxide:

According to one embodiment, the invention relates to a cosmetic treatment process for improving the surface appearance of the skin, and in particular for reducing visible and/or tactile skin irregularities, for instance the cutaneous microrelief, which consists in applying to said irregularities a filling product resulting from a crosslinking reaction in the presence of a peroxide, between two compounds X and Y, at least one of which is a silicone compound, said compounds being placed in contact with each other in the presence of a peroxide, such that said reaction takes place at least partly on the skin.

This reaction is preferably performed by heating to a temperature of greater than or equal to 50° C., preferably greater than or equal to 80° C., which may be up to 120° C.

The identical or different compounds X and Y comprise in this case at least two —CH₃ side groups and/or at least two side chains bearing a —CH₃ group.

Compounds X and Y are preferably silicone compounds and may be chosen, for example, from high molecular weight non-volatile linear polydimethylsiloxanes, with a degree of polymerization of greater than 6, containing at least two —CH₃ side groups bonded to the silicon atom and/or at least two side chains bearing a —CH₃ group. Mention may be made, for example, of polymers described in the “Reactive Silicones” catalogue from the company Gelest Inc., Edition 2004, page 6, and especially vinylmethylsiloxane-dimethylsiloxane copolymers (also referred to as gums) with molecular weights ranging from 500 000 to 900 000 and a viscosity of greater than 2 000 000 cSt.

As peroxides that may be used in the context of the invention, mention may be made of benzoyl peroxide and 2,4-dichlorobenzoyl peroxide, and mixtures thereof.

According to one embodiment, the hydrosilylation reaction or the condensation reaction, or alternatively the crosslinking reaction in the presence of a peroxide, between compounds X and Y is accelerated by supplying heat, for example by raising the temperature of the system to between 25° C. and 180° C.

In general, irrespective of the type of reaction via which compounds X and Y react together, the mole percentage of X relative to all of the compounds X and Y, i.e. the ratio X/(X+Y)×100, may range from 5% to 95%, preferably from 10% to 90% and better still from 20% to 80%.

Similarly, the mole percentage of Y relative to all of the compounds X and A, i.e. the ratio Y/(X+Y)×100, may range from 5% to 95%, preferably from 10% to 90% and better still from 20% to 80%.

Compound X may have a weight-average molecular mass (Mw) ranging from 150 to 1 000 000, preferably from 200 to 800 000 and more preferably from 200 to 250 000.

Compound Y may have a weight-average molecular mass (Mw) ranging from 200 to 1 000 000, preferably from 300 to 800 000 and more preferably from 500 to 250 000.

Compound X may represent from 0.1% to 95% by weight, preferably from 1% to 90% and better still from 5% to 80% by weight relative to the total weight of the composition.

Compound Y may represent from 0.1% to 95% by weight, preferably from 1% to 90% and better still from 5% to 80% by weight relative to the total weight of the composition.

The ratio between the compounds X and Y may be varied so as to modify the rate of reaction and thus the rate of formation of the film, or alternatively so as to adapt the properties of the film formed (for example its adhesive properties) according to the desired application.

In particular, compounds X and Y may be present in a mole ratio X/Y ranging from 0.05 to 20 and better still from 0.1 to 10.

Compounds X and Y may advantageously be combined with at least one filler. Thus, the kit according to the invention may, for example, comprise, in at least one of the compositions, a filler chosen from silica and surface-treated silica.

As stated previously, according to one embodiment of the invention, compounds X and Y may be used in the form of a single composition, which then contains at least one of them or, where appropriate, the catalyst or the peroxide, if necessary for their interaction, in an encapsulated form.

In the context of the present invention, encapsulated forms of core/shell type, also known as microcapsules, or nanocapsules, the shell of which is of polymeric nature and the core of which contains compound X or compound Y, are more particularly considered, one of its compounds X and Y being, where appropriate, encapsulated with the catalyst or the peroxide if necessary for interaction of the two compounds, When this catalyst is not encapsulated with one or the other of the compounds X or Y, it is present in the cosmetic composition containing the encapsulated forms.

Many techniques are nowadays available for preparing microcapsules or nanocapsules of this type.

However, according to one preferred mode, the encapsulated forms under consideration according to the invention are nanocapsules and are obtained via a “solvent tipping” technique illustrated especially in documents BP 274 961 and EP 1 552 820.

More particularly, the shell of the nanocapsules of compound X or Y used according to the invention is of polymeric, non-crosslinked nature, and is water-insoluble and insoluble in the core of the capsules.

In general, any polymer, of natural or synthetic origin, which is soluble in a water-immiscible solvent, and especially those with a melting point lower than the boiling point of water at atmospheric pressure (100° C.), may be suitable for use.

These polymers may be biodegradable, for instance polyesters, or non-biodegradable.

As illustrations of polymers that are suitable for use in the invention, mention may especially be made of:
C₂-C₁₂ alkyl cyanoacrylate polymers,
polymers formed from poly-L-lactides, poly-DL-lactides, polyglycolides and the corresponding copolymers,
polycaprolactones,
3-hydroxybutyric acid polymers,
copolymers of vinyl chloride and of vinyl acetate,
copolymers of methacrylic acid and ester, especially of methacrylic acid and of methacrylic acid ester,
polyvinyl acetophthalate,
cellulose acetophthalate,
polyvinylpyrrolidone/vinyl acetate copolymer,
poly(ethylene/vinyl acetates),
polyacrylonitriles,
polyacrylamides,
polyethylene glycols,
poly(C₁-C₄ hydroxyalkyl methacrylates),
cellulose esters of C₁-C₄ carboxylic acids,
polystyrene and copolymers of styrene and of maleic anhydride, copolymers of styrene and of acrylic acid, styrene/ethylene/butylene/styrene block terpolymers and styrene/ethylene/propylene/styrene block terpolymers,
styrene/alkyl alcohol oligomers,
terpolymers of ethylene, of vinyl acetate and of maleic anhydride,
polyamides,
polyethylenes,
polypropylenes,
organopolysiloxanes, including polydimethylsiloxanes, poly(alkylene adipates),
the polyester polyols,
polysilsesquioxane silicone polymers,
dendritic polyesters containing a hydroxyl end function,
polymers that are water-dispersible but soluble in water-immiscible solvents, for instance: polyesters, poly(ester amides), polyurethanes and vinyl copolymers bearing carboxylic acid and/or sulfonic acid functions, and in particular those described in document FR 2 787 729,
block copolymers that are insoluble in water at room temperature and solid at room temperature, containing at least one block of one of the above polymers, and
mixtures thereof.

These polymers or copolymers may have a weight-average molecular weight of between 1000 and 500 000 and in particular between 1500 and 100 000.

Poly(alkylene adipates), organopolysiloxanes, polycaprolactones, cellulose acetophthalate, cellulose acetobutyrate, cellulose esters, and polystyrene and derivatives thereof, are most particularly suitable for the invention.

Needless to say, a person skilled in the art is capable, on the basis of his knowledge, of adjusting the molecular weight of the selected polymer with respect to its concentration in the solvent in order to have a viscosity of the mixture that is compatible with satisfactory emulsification.

As regards the lipophilic core, it may contain, besides compound X or compound y, at least one oil. The oil may be chosen from the oils described hereinbelow for the oily phase. The oil is preferably a silicone oil.

According to one variant of the invention, the encapsulated forms of compound X or compound Y may be coated with a lamellar phase.

As regards the procedure for preparing nanocapsules that are suitable for use in the invention, a person skilled in the art may refer especially to the teaching of document EP 1 552 820 cited previously. The choice of the required surfactants and the implementation of the process call upon the knowledge of a person skilled in the art.

### Fillers

The kits according to the invention, the compositions according to the invention and the compositions of the cosmetic process according to the invention may comprise at least one filler whose nature and amount are such that the filling product obtained after the interaction of X and Y is opaque.

In particular, at least one of the compositions from which the filling product used in a process in accordance with the invention is derived comprises at least one filler.

These fillers may also advantageously make it possible to reinforce the physical filling effect of facial wrinkles achieved by the interaction of compounds X and Y according to the invention.

As fillers that may be used according to the invention, mention may be made especially of:
silica and especially porous silica microparticles, for instance the silica beads SB150 and SB700® from Miyoshi with a mean size of 5 microns; the series-H Sunspheres from Asahi Glass, for instance Sunspheres H33®, H51® and H53® with respective sizes of 3, 5 and 5 microns;
polytetrafluoroethylene (PTFE) powders, for instance the PTFE Ceridust 9205F® from Clariant, with a mean size of 8 microns;
silicone resin powders, for instance the silicone resin Tospearl 145A® from GE Silicone, with a mean size of 4.5 microns;
hollow hemispherical silicone particles such as NLK 500, NLK 506 and NLK 510 from Takemoto Oil and Fat;
acrylic copolymer powders, especially of polymethyl (meth)acrylate, for instance the PMMA particles Jurymer MBI® from Nihon Junyoki, with a mean size of 8 microns, the hollow PMMA spheres sold under the name Covabead LH85® by the company Wacker, and the vinylidene chloride/acrylonitrile/methylene methacrylate expanded microspheres sold under the name Expancel®;
polyethylene powders, especially comprising at least one ethylene/acrylic acid copolymer, and in particular consisting of ethylene/acrylic acid copolymers, for instance the polyethylene AC540® particles or the Flobeads EA 209® particles from Sumitomo, with a mean size of 10 microns;
crosslinked elastomeric organopolysiloxane powders coated with silicone resin and especially with silsesquioxane resin, as described, for example, in U.S. Pat. No. 5,538,793. Such elastomer powders are sold under the names KSP-100®, KSP-100®, KSP-102®, KSP-103®, KSP-104® and KSP-105® by the company Shin-Etsu;
mixtures of crosslinked polydimethylsiloxane and of polydimethylsiloxane, sold under the names KSG-6® and KSG-16® by the company Shin-Etsu;
talc/titanium dioxide/alumina/silica composite powders, for instance those sold under the name Coverleaf AR-80® by the company Catalyst & Chemicals;
polyamide (Nylon®) powders, for instance the Nylon 12 particles of the Orgasol® type from Atofma, with a mean size of 10 microns;
expanded powders, such as hollow microspheres and especially microspheres formed from a terpolymer of vinylidene chloride, of acrylonitrile and of methacrylate and sold under the name Expancel® by the company Kemanord Plast under the references 551 DE 12® (particle size of about 12 μm and mass per unit volume of 40 kg/m³), 551 DE 20® (particle size of about 30 μm and mass per unit volume of 65 kg/m³), 551 DE 50® (particle size of about 40 μm),
the microspheres sold under the name Micropearl F 80 ED® by the company Matsumoto;
powders of natural organic materials such as starch powders, especially of crosslinked or non-crosslinked corn starch, wheat starch or rice starch, such as starch powders crosslinked with octenyl succinate anhydride, sold under the name Dry-Flo® by the company National Starch;
and mixtures thereof.

The filler may preferably be chosen from silica, porous silica microparticles, silicone resin powders, polyethylene powders, mixtures of crosslinked polydimethylsiloxane and of polydimethylsiloxane, and expanded powders, and mixtures thereof.

The amount of fillers introduced obviously depends on the desired effect, but they may generally represent from 0.1% to 35% by weight and preferably from 0.5% to 20% by weight relative to the total weight of the product, of each of the compositions or of the sum of the weights of the first and second compositions containing them.

Advantageously, the amount of fillers in the filling product may range from 10% to 35% by weight and preferably from 10% to 20% by weight relative to the total weight of said product.

### Waxes

According to one preferred mode of the invention, the product, the composition or one of the first and second compositions, especially when it is in the form of a paste, may comprise a wax.

In particular, at least one of the compositions from which the filling product used in a process in accordance with the invention is derived also comprises at least one wax.

For the purposes of the present invention, a wax is a lipophilic fatty compound, which is solid at room temperature (25° C.), with a reversible solid/liquid change of state, which has a melting point of greater than about 45° C. (measured by DSC) and better still greater than 50° C., which may be up to 200° C., and which has, in the solid state, an anisotropic crystal organization.

For the purposes of the patent application, the waxes are generally those used in cosmetics and dermatology; they may be hydrocarbon-based waxes, silicone waxes and/or fluoro waxes, optionally comprising ester or hydroxyl functions.

As waxes that may be used in the composition of the invention, examples that may be mentioned include mineral waxes such as microcrystalline waxes, paraffin, petroleum jelly, ozokerite and montan wax; waxes of animal origin such as beeswax, and lanolin and its derivatives; waxes of plant origin such as candelilla wax, ouricury wax, carnauba wax, Japan wax, cocoa butter, cork fiber wax or sugarcane wax; hydrocarbon-based oils that are solid at 25° C.; fatty esters and glycerides that are solid at 25° C.; synthetic waxes such as polyethylene waxes and the waxes obtained by Fischer-Tropsch synthesis; silicone waxes, and mixtures thereof.

According to one preferred embodiment of the invention, at least one wax with a starting melting point of greater than or equal to 50° C., and better still at least one wax whose starting melting point is greater than 65° C., is used, such as carnauba wax, certain polyethylene waxes and certain microcrystalline waxes such as the wax sold under the name Microwax®, in particular Microwax HW®, by the company Paramelt.

In the present patent application, the term “starting melting point” means the temperature at which a wax begins to melt. This temperature may be determined by DTA (differential thermal analysis), which makes it possible to obtain the thermogram (or melting curve) of the wax under consideration. The starting melting point corresponds to the temperature at which a noticeable change of gradient may be observed in the thermogram. The melting point, itself, represents the minimum point of said thermogram.

In order to effectively incorporate the waxes and fillers that may be present, it is advantageous to perform the preparation of the composition or at least one step of this preparation, in particular the preparation of the oily phase or one step of preparation of the oily phase, in a mixer-screw extruder, subjected to a temperature gradient ranging from 100° C. to 20° C. It is in particular advantageous to prepare the mixture of fillers, of waxes and of at least one oil, in a mixer-screw extruder, subjected to a temperature gradient ranging from 100° C. to 20° C. Such a preparation is especially described in patent applications EP 1 005 856, EP 1 005 857 and EP 1 013 267.

The wax may be present in a content ranging from 0.1% to 30% by weight, especially from 5% to 30% by weight, preferably from 1% to 15% by weight and especially from 5% to 15% by weight relative to the total weight of the product, of each of the compositions or of the sum of the weights of the first and second compositions containing it.

According to one advantageous embodiment, the product according to the invention or the compositions according to the invention, and the compositions of the cosmetic process according to the invention, may comprise at least one anti-aging active agent having at least one preventive and/or curative effect on at least one sign of aging of the skin. These compounds are active agents that act on the epidermis and/or the dermis.

As anti-aging active agents that may be used according to the invention, mention may be made especially of any active agent known for its activity on aging of the skin, for instance α-hydroxy acids (for example lactic acid), β-hydroxy acids (for example salicylic acid), retinoids and esters thereof, retinal, retinoic acid and its derivatives (for example retinyl palmitate or retinyl acetate), adenosine and its derivatives, ascorbic acid and its derivatives, and C-glycosides and derivatives thereof (especially those described in patent application WO 02/051 828) and in particular C-α-D-xylopyranoside-2-hydroxypropane.

Vitamins may also be mentioned, for instance vitamins B3, PP, B5, E or K1.

The amount of anti-aging active agents obviously depends on the nature of the active agent and on the desired effect, but this amount generally represents from 0.01% to 10% by weight and preferably from 0.1% to 5% by weight relative to the total weight of each of the compositions or of the sum of the weights of the first and second compositions.

Preferably, the anti-aging active agent is chosen from adenosine and its derivatives, ascorbic acid and its derivatives, and C-glycosides and derivatives thereof, for instance C-α-D-xylopyranoside-2-hydroxypropane.

As indicated previously, the kit according to the invention, the compositions according to the invention and the compositions of the process according to the invention may also advantageously comprise interference particles, for instance small nacres or interference pigments.

Specifically, such interference particles may, in combination, give skin, coated with a film in accordance with the invention, an additional effect of lightening, unifying or even concealing nature with regard to skin imperfections. Thus, by virtue of their presence, they advantageously make it possible to reinforce the visual perception of the improvement in the surface appearance of the skin afforded by the compositions under consideration according to the invention.

In particular, the presence of interference particles may make it possible to afford an additional concealing effect such that the visual perception of visible and/or tactile skin irregularities, for instance the cutaneous microrelief, is found to be attenuated.

When combined with fillers, which may be particulate fillers, the interference particles according to the invention may in particular make it possible to obtain a transparency effect such that the effect afforded by the corresponding composition on the skin virtually does not affect, or does not at all affect, the natural flesh tone of the skin.

For the purposes of the present invention, the term “interference particles” denotes any particle generally having a multilayer structure such that it allows the creation of a color effect by interference of light rays that diffract and diffuse differently according to the nature of the layers. Thus, these particles may have colors that vary according to the angle of observation and the incidence of the light.

The color effects obtained are associated with the multilayer structure of these particles and are derived from the physical laws of optics of thin layers (see: Pearl Lustre Pigments—Physical principles, properties, applications—R. Maisch, M. Weigand, Verlag Moderne Industrie).

For the purposes of the present invention, a multilayer structure is intended to denote either a structure formed from a substrate covered with a single layer or a structure formed from a substrate covered with at least two or even more consecutive layers.

The multilayer structure may thus comprise one or even at least two layers, each layer, independently or otherwise of the other layer(s), being made of at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys and polymers, and combinations thereof.

Generally, the multilayer structure is of mineral nature.

More particularly, the interference particles under consideration according to the invention may be interference pigments, or alternatively natural or synthetic, monolayer or multilayer nacres, in particular formed from a natural substrate based, inter alia, on mica, which is covered with one or more layers of metal oxide.

The interference particles according to the invention are characterized by a volumetric mean size generally of less than 40 μm, especially ranging from 0.5 to 40 μm, more particularly less than 30 μm, especially less than 20 μm, in particular less than 15 μm and better still ranging from 1 to 15 μm, measured with a laser granulometer, for instance the Mastersizer 2000® machine from Malvern and/or the BI90+® machine from Brookhaven Instrument Corporation.

The nacres preferably chosen from mica/tin oxide/titanium oxide nacres, for instance those sold under the names Timiron Silk Blue®, Timiron Silk Red®, Timiron Silk Green®, Timiron Silk Gold® and Timiron Super Silk® sold by the company Merck, and mica/iron oxide/titanium oxide nacres, for instance Flamenco Satin Blue®, Flamenco Satin Red® and Flamenco Satin Violet® sold by the company Engelhard, and mixtures thereof, are most particularly suitable for use in the invention.

It is understood that the choice of these interference particles is made so as to be moreover compatible with the requirements in terms of lightness and saturation required for the compositions according to the invention. In general, these interference particles are present in an amount sufficient to obtain a homogeneous effect in terms of coloration while at the same time preserving the natural flesh tone of the skin and/or the lips.

More precisely, these particles may represent from 0.1% to 15% by weight, more particularly from 0.1% to 7% by weight and more particularly from 0.1% to 5% by weight relative to the total weight of the product, of each of the compositions or of the sum of the weights of the first and second compositions.

### Physiologically Acceptable Medium

As stated previously, the compositions according to the invention comprise a physiologically acceptable medium, i.e. a nontoxic medium of pleasant appearance, odor and feel, which may be applied to human keratin materials.

The compositions according to the invention advantageously contain at least one liquid fatty phase.

The compositions according to the invention may be in the form of anhydrous compositions or emulsions.

Advantageously, they may be in anhydrous form.

For the purposes of the invention, the term “anhydrous composition” denotes a composition that contains less than 2% by weight of water, or even less than 0.5% water relative to its total weight, and especially a water-free composition.

The compositions according to the invention may also be in the form of an emulsion obtained by dispersing an aqueous phase in a fatty phase (W/O) or a fatty phase in an aqueous phase (O/W), of liquid or semi-liquid consistency of the milk type, or of soft, semi-solid or solid consistency of the cream or gel type, or alternatively a multiple emulsion (W/O/W or O/W/O). These compositions are prepared according to the usual methods.

The weight ratio between the aqueous phase and the oily phase may be, for example, from 10/90 to 95/5 and preferably from 30/70 to 90/10. Thus, the aqueous phase may represent, for example, from 10% to 99% by weight, preferably from 30% to 98% by weight and better still from 60% to 95% by weight relative to the total weight of the compositions according to the invention, and the oily phase may represent, for example, from 1% to 90% by weight, preferably from 2% to 70% by weight and better still from 5% to 40% by weight relative to the total weight of the compositions according to the invention.

The aqueous phase may also comprise organic solvents that are water-miscible (at 25° C.), for instance primary alcohols (C₁-C₃ monohydric alcohols) such as ethanol and isopropanol, polyols such as propylene glycol, butylene glycol, glycerol and hexylene glycol, polyethylene glycols such as PEG-8 and dipropylene glycol, and mixtures thereof. The amount of polyol(s) generally ranges from 0.05% to 20% by weight, preferably from 0.1% to 15% by weight and better still from 0.5% to 10% by weight relative to the total weight of the compositions according to the invention.

The emulsions generally contain at least one emulsifier chosen from amphoteric, anionic, cationic and nonionic emulsifiers, used alone or as a mixture. The emulsifiers are chosen in a suitable manner according to the emulsion to be obtained (W/O or O/W). The emulsifiers are generally present in the composition in a proportion that may range, for example, from 0.3% to 30% by weight and preferably from 0.5% to 20% by weight relative to the total weight of the composition.

As examples of oils that may be used in the composition according to the invention, mention may be made of:
hydrocarbon-based oils of animal origin, such as perhydrosqualene;
hydrocarbon-based oils of plant origin, such as liquid triglycerides of fatty acids of 4 to 10 carbon atoms, such as heptanoic or octanoic acid triglycerides or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, caprylic/capric acid triglycerides such as those sold by the company Stéarineries Dubois or those sold under the names Miglyol 810, 812 and 818 by the company Dynamit Nobel, jojoba oil or shea butter oil;
synthetic esters and ethers in particular of fatty acids, such as the oils of formulae R₁COOR₂ and R₁OR₂ in which R₁, represents a fatty acid residue containing from 8 to 29 carbon atoms and R₂ represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactater octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;
linear or branched hydrocarbons of mineral or synthetic origin, such as volatile or nonvolatile liquid paraffins and derivatives thereof, isohexadecane, isododecane, petroleum jelly, polydecenes or hydrogenated polyisobutene such as Parleam Oil®;
natural or synthetic essential oils such as, for example, eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;
fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils such as those described in document JP-A-2-295 912;
silicone oils such as volatile or nonvolatile polydimethylsiloxanes (PDMSs) containing a linear or cyclic silicone chain, which are liquid or pasty at room temperature, in particular cyclopolydimethyl-siloxanes (cyclomethicones) such as cyclohexasiloxane; polydimethylsiloxanes comprising alkyl, alkoxy or phenyl groups, pendant or at the end of a silicone chain, these groups containing from 2 to 24 carbon atoms; phenyl silicones such as phenyl trimethicones, phenyl dimethicones, phenyltrimethylsiloxydiphenyl-siloxanes, diphenyl dimethicones, diphenylmethyl-diphenyltrisiloxanes, 2-phenylethyl trimethylsiloxy-silicates and polymethylphenylsiloxanes;
mixtures thereof,

The term “hydrocarbon-based oil” in the list of oils mentioned above means any oil predominantly comprising carbon and hydrogen atoms, and optionally ester, ether, fluoro, carboxylic acid and/or alcohol groups.

The compositions according to the invention may comprise a volatile oil.

For the purposes of the invention, the term “volatile oil” means an oil that is capable of evaporating on contact with keratin materials in less than one hour, at room temperature and atmospheric pressure. The volatile oils of the invention are volatile cosmetic oils that are liquid at room temperature, with a nonzero vapor pressure, at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile oils that may be mentioned, inter alia, include cyclic or linear silicones containing from 2 to 6 silicon atoms, such as cyclohexasiloxane, dodecamethylpentasiloxane, decamethyltetrasiloxane, butyl trisiloxane and ethyl trisiloxane. It is also possible to use branched hydrocarbons, for instance isododecane, and also volatile perfluoroalkanes such as dodecafluoropentane and tetradecafluorohexane, sold under the names PF 5050® and PF 5060® by the company 3M, and perfluoro morpholine derivatives, such as 4-trifluoromethylperfluoromorpholine sold under the name PF 5052® by the company 3M.

The amount of oily phase present in the compositions according to the invention may range, for example, from 0.01% to 50% by weight and preferably from 0.1% to 30% by weight relative to the total weight of the composition.

The compositions according to the invention may also comprise at least one dyestuff chosen, for example, from pigments, nacres, dyes and materials with an effect, and mixtures thereof.

These dyestuffs may be present in a content ranging from 0.01% to 50% by weight and preferably from 0.01% to 30% by weight relative to the total weight of the composition.

The composition according to the invention may also contain various adjuvants commonly used in cosmetics, such as sequestrants; fragrances; and thickeners and gelling agents. The amounts of these various adjuvants and the nature thereof will be chosen such that they do not harm the optical properties of the composition.

Depending on the fluidity of the composition that it is desired to obtain, one or more gelling agents, especially hydrophilic gelling agents, i.e. water-soluble or water-dispersible gelling agents, or alternatively lipophilic gelling agents, may be incorporated into the composition.

Hydrophilic gelling agents that may be mentioned in particular include water-soluble or water-dispersible thickening polymers. These polymers may be chosen especially from: modified or unmodified carboxyvinyl polymers, such as the products sold under the names Carbopol (CTFA name: carbomer) and Pemulen (CTFA name: Acrylate/_{C10-30} alkyl acrylate crosspolymer by the company Goodrich; polyacrylates and polymethacrylates such as the products sold under the names Lubrajel and Norgel by the company Guardian or under the name Hispagel by the company Hispano Chimica; polyacrylamides; optionally crosslinked and/or neutralized 2-acrylamido-2-methylpropane sulfonic acid polymers and copolymers, for instance the poly(2-acrylamido-2-methylpropanesulfonic acid) sold by the company Clariant under the name Hostacerin AMPS (CTFA name: ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C₁₃₋₁₄ Isoparaffin/Laureth-7) and under the name Simulgel 600 (CTFA name: Acrylamide/Sodium acryloyldimethyltaurate copolymer/Isohexadecane/Polysorbate 80) by the company SEPPIC, polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose; and mixtures thereof.

Examples of lipophilic gelling agents that may also be mentioned include modified clays such as modified magnesium silicate (Bentone gel VS38 from Rheox), hectorite modified with distearyldimethylammonium chloride (CTFA name: distearate diammonium hectorite) sold under the name Bentone 38 CE by the company Rheox, or silicone elastomers such as those sold under the names KSG-6 or KSG-16 by the company Shin-Etsu.

The compositions containing compounds X and Y according to the invention may also contain other conventional cosmetic or care adjuvants or additives, for instance active agents, preserving agents, antiseptics, bactericides, softeners, moisturizers, for instance polyols, buffers, humectants, UV-screening agents (or sunscreens), bleaching and/or depigmenting agents, cosmetic or dermatological active agents, free-radical scavengers, electrolytes such as sodium chloride or a pH modifier, for example citric acid or sodium hydroxide, and also mixtures thereof.

Needless to say, a person skilled in the art will take care to select this or these optional additives(s) and/or the amount thereof such that the advantageous properties of the compositions according to the invention are not, or are not substantially, adversely affected by the envisaged addition.

The invention is illustrated in greater detail by the example of a filling product described hereinbelow.

In the composition examples described hereinbelow, the combination of mixtures A and B below prepared by the company Dow Corning is used as compounds X and Y:

| | | | |
|---|---|---|---|
| Ingredient (INCI name) | CAS No. | Content (%) | Function |

MIXTURE A:

| | | | |
|---|---|---|---|
| Dimethylsiloxane, | 68083-19-2 | 55-95 | Polymer |
| Dimethylvinylsiloxy- | | | |
| terminated | | | |
| Silica Silylate | 68909-20-6 | 10-40 | Filler |
| 1,3-Diethenyl-1,1,3,3-Tetra- | 68478-92-2 | Trace | Catalyst |
| methyldisiloxane complexes | | | |
| Tetramethyldivinyldisiloxane | 2627-95-4 | 0.1-1 | Polymer |

MIXTURE B:

| | | | |
|---|---|---|---|
| Dimethylsiloxane, | 68083-19-2 | 55-95 | Polymer |
| Dimethylvinylsiloxy- | | | |
| terminated | | | |
| Silica Silylate | 68909-20-6 | 10-40 | Filler |
| Dimethyl, | 68037-59-2 | 1-10 | Polymer |
| Methylhydrogenosiloxane, | | | |
| trimethylsiloxy-terminated | | | |

### EXAMPLE

### Example 1

### Cosmetic Filling Product

First composition

| | |
|---|---|
| Mixture of dimethicone/vinyl dimethicone | 13.20 g |
| copolymer and of dimethicone (KSG-6) | |
| Acrylate copolymer (Expancel 551 DE 40 D42) | 0.15 g |
| Microcrystalline waxes (Microwax HW) | 6.00 g |
| Polydimethyl silsesquioxane (Tospearl 145A) | 2.00 g |
| Parleam | 23.00 g |
| Porous particles containing 10% adenosine* | 2.50 g |
| Acrylic acid/ethylene copolymer (Polyethylene AC 540) | 3.00 g |
| Preserving agent | 0.15 g |
| Mixture A | 50.00 g |

*The filled polyamide particles are prepared in the following manner: a solution containing 2 g of adenosine, 100 ml of water, 0.5 g of an ethylene oxide/propylene oxide/ethylene oxide copolymer (128 EO/54 PO/128 EO - Poloxamer 338 from Uniqema) and 5 g of 1N hydrochloric acid solution is prepared. This solution is stirred at room temperature until dissolution of the active agent is complete. 38 g of Nylon-12 (Orgasol 2002 D Nat Cos 204 from Arkema) are then dispersed in this solution, and the dispersion is then transferred into a round-bottomed flask and the water is evaporated off on a rotavapor at 40° C. for 8 hours. After total evaporation of the solvent, a powder constituted of Nylon particles whose pores are filled with adenosine in solid form is thus obtained.

Second composition

| | |
|---|---|
| Mixture of dimethicone/vinyl dimethicone | 13.2 g |
| copolymer and of dimethicone (KSG-6) | |
| Acrylate copolymer (Expancel 551 DE 40 D42) | 0.15 g |
| Microcrystalline waxes (Microwax HW) | 6.00 g |
| Polydimethyl silsesguioxane (Tospearl 145A) | 2.00 g |
| Parleam | 23.00 g |
| Porous particles containing 10% adenosine* | 2.50 g |
| Acrylic acid/ethylene copolymer (Polyethylene AC 540) | 3.00 g |
| Preserving agent | 0.15 g |
| Mixture B | 50.00 g |

*as described for the first composition

The first and second compositions are mixed together extemporaneously in a 50/50 weight proportion.

This mixture is then applied to the wrinkles and fine lines of the crow's feet and around the crow's feet of a panel of women with mature skin, for whom a substantial reduction in the wrinkles and fine lines is subsequently observed.

### Example 2

### Cosmetic Filling Product

First composition

| | |
|---|---|
| Mixture of dimethicone/vinyl dimethicone | 13.20 g |
| copolymer and of dimethicone (KSG-6) | |
| Acrylate copolymer (Expancel 551 DE 40 D42) | 0.15 g |
| Microcrystalline waxes (Microwax HW) | 4.00 g |
| Mica/titanium oxide/tin oxide nacres (Timiron | 2.00 g |
| Silk Red ® from Merck) | |
| Polydimethyl silsesquioxane (Tospearl 145A) | 2.00 g |
| Parleam | 23.00 g |
| Porous particles containing 10% adenosine* | 2.50 g |
| Acrylic acid/ethylene copolymer (Polyethylene AC 540) | 3.00 g |
| Preserving agent | 0.15 g |
| Mixture A | 50.00 g |

*as described for the first composition of Example 1

Second composition

| | |
|---|---|
| Mixture of dimethicone/vinyl dimethicone | 13.2 g |
| copolymer and of dimethicone (KSG-6) | |
| Acrylate copolymer (Expancel 551 DE 40 D42) | 0.15 g |
| Microcrystalline waxes (Microwax HW) | 4.00 g |
| Mica/titanium oxide/tin oxide nacres (Timiron | 2.00 g |
| Silk Red ® from Merck) | |
| Polydimethyl silsesquioxane (Tospearl 145A) | 2.00 g |
| Parleam | 23.00 g |
| Porous particles containing 10% adenosine* | 2.50 g |
| Acrylic acid/ethylene copolymer (Polyethylene AC 540) | 3.00 g |
| Preserving agent | 0.15 g |
| Mixture B | 50.00 g |

*as described for the first composition of Example 1

the first and second compositions are mixed together extemporaneously in a 50/50 weight proportion.

This mixture is then applied to the wrinkles and fine lines of the crow's feet and around the crow's feet of a panel of women with mature skin, for whom a substantial reduction in the wrinkles and fine lines is subsequently observed.

The presence of interference particles enhances the visual perception of the reduction of the wrinkles that is achieved by this filling product.

1-44. (canceled)

45. A cosmetic treatment process for improving the surface appearance of the skin, which consists in applying to said irregularities a filling product resulting from a hydrosilylation reaction in the presence of a catalyst, between two compounds X and Y, at least one of which is a silicone compound, said compounds being placed in contact with each other in the presence of a catalyst, such that said reaction takes place at least partly on the skin.

46. The process as claimed in claim 45, wherein the filling product is obtained by extemporaneously mixing at least one first composition comprising, in a physiologically acceptable medium, at least said compound X, and one second composition comprising, in a physiologically acceptable medium, at least said compound Y, with at least one of said first and second compositions also containing at least one catalyst.

47. A cosmetic treatment process for improving the surface appearance of the skin comprising at least the application to skin presenting said irregularities of a filling product resulting from the application of at least:
one coat of a first composition comprising, in a physiologically acceptable medium, at least one compound X, and
one coat of a second composition comprising, in a physiologically acceptable medium, at least one compound Y,
at least one of the compounds X and Y being a silicone compound, said compounds X and Y being capable of reacting together via a hydrosilylation reaction in the presence of a catalyst, when they are placed in contact with each other, with at least one of said first and second compositions also containing at least one catalyst.

48. The process as claimed in claim 47, wherein several coats of each of the first and second compositions are applied alternately to the skin.

49. The process as claimed in claim 45, also comprising the application of interference particles to said irregularities.

50. The process as claimed in claim 46, wherein at least one of the compositions from which the filling product is derived is anhydrous.

51. The process as claimed in claim 46, wherein both compositions are anhydrous.

52. The process as claimed in claim 46, wherein at least one of the compositions from which the filling product is derived comprises at least one filler.

53. The process as claimed in claim 50, wherein said filler is chosen from silica, polytetrafluoroethylene powders, silicone resin powders, hollow hemispherical silicone particles, acrylic copolymer powders, wax powders, polyethylene powders, crosslinked elastomeric organopolysiloxane powders coated with silicone resin, mixtures of crosslinked polydimethylsiloxane and of polydimethylsiloxane, composite powders, polyamide powders, expanded powders, microspheres, and powders of natural organic materials, and mixtures thereof.

54. The process as claimed in claim 53, wherein said filler is chosen from silica, silicone resin powders, polyethylene powders, mixtures of crosslinked polydimethylsiloxane and of polydimethylsiloxane, and expanded powders, and a mixture thereof.

55. The process as claimed in claim 52, wherein said filler represents from 0.1% to 35% by weight relative to the total weight of the composition containing it.

56. The process as claimed in claim 45, wherein at least one of the compositions from which the filling product is derived also contains at least one wax.

57. The process as claimed in claim 56, wherein the wax represents from 0.1% to 30% by weight relative to the total weight of the composition containing it.

58. The process as claimed in claim 45, wherein said compound X is chosen from silicone compounds comprising at least two unsaturated aliphatic groups.

59. The process as claimed in claim 58>wherein said compound X is a polyorganosiloxane comprising a main silicone chain whose unsaturated aliphatic groups are pendent on the main chain (side group) or located at the ends of the main chain of the compound (end group).

60. The process as claimed in claim 59, wherein said compound X bears at least one polar group.

61. The process as claimed in claim 45, wherein said compound X is chosen from polyorganosiloxanes comprising at least two unsaturated aliphatic groups each bonded to a silicon atom.

62. The process as claimed in claim 45, wherein said compound X is chosen from polyorganosiloxanes comprising siloxane units of formula:
$${R}_{m} {R}^{′} {SiO}_{\frac{( 3 - m )}{2}} \qquad ( I )$$
in which:
R represents a linear or cyclic monovalent hydrocarbon-based group containing from 1 to 30 carbon atoms,
m is equal to 1 or 2, and
R′ represents:
an unsaturated aliphatic hydrocarbon-based group containing from 2 to 10 carbon atoms, or
an unsaturated cyclic hydrocarbon-based group containing from 5 to 8 carbon atoms.

63. The process as claimed in claim 62, wherein the polyorganosiloxane of formula (I) is such that R′ represents a vinyl group or a group —R″—CH═CHR′ in which R″ is a divalent aliphatic hydrocarbon-based chain containing from 1 to 8 carbon atoms, bonded to the silicon atom and R′ is a hydrogen atom or an alkyl radical containing from 1 to 4 carbon atoms.

64. The process as claimed in claim 62, wherein R represents an alkyl radical containing from 1 to 10 carbon atoms or a phenyl group, and R′ is a vinyl group.

65. The process as claimed in claim 63, wherein the polyorganosiloxanes also comprise units of formula:
$${R}_{n} {SiO}_{\frac{( 4 - n )}{2}} \qquad ( II )$$
in which R is a linear or cyclic monovalent hydrocarbon-based group containing from 1 to 30 carbon atoms, and n is equal to 1, 2 or 3.

66. The process as claimed in claim 45, wherein said compound X is chosen from organic oligomers or polymers and organic/silicone hybrid oligomers or polymers, said oligomers or polymers bearing at least two reactive unsaturated aliphatic groups.

67. The process as claimed in claim 45, wherein said compound Y comprises at least two free Si—H groups.

68. The process as claimed in claim 45, wherein said compound Y is chosen from polyorganosiloxanes comprising at least one alkylhydrogenosiloxane unit having the following formula:
$${R}_{p} {HSiO}_{\frac{( 3 - p )}{2}} \qquad ( III )$$
in which:
R represents a linear or cyclic monovalent hydrocarbon-based group containing from 1 to 30 carbon atoms, or a phenyl group, and p is equal to 1 or 2.

69. The process as claimed in claim 68, wherein said compound Y is such that the radicals R represents a C₁-C₁₀ alkyl group.

70. The process as claimed in claim 45, wherein Y is a polyorganosiloxane comprising at least two alkylhydrogenosiloxane units of formula —(H₃C)(H)Si—O— and optionally comprising units —(H₃C)₂SiO—.

71. The process as claimed in claim 45, wherein said catalyst is a platinum-based or tin-based catalyst.

72. The process as claimed in claim 71, wherein said catalyst is present in a content ranging from 0.0001% to 20% by weight relative to the total weight of the composition comprising it.

73. The process as claimed in claim 45, wherein said compound X is a polydimethylsiloxane containing vinyl end groups and compound Y is a polymethylhydrogenosiloxane.

74. The process as claimed in claim 45, in which compound X bears at least one polar group capable of forming a hydrogen bond with keratin materials.

75. The process as claimed in claim 46, comprising, in at least one of the compositions from which the filling product is derived, a filler chosen from silica and surface-treated silica.

76. The process as claimed in claim 45, wherein said compound X has a weight-average molecular mass (Mw) ranging from 150 to 1 000 000.

77. The process as claimed in claim 45, wherein said compound Y has a weight-average molecular mass (Mw) ranging from 200 to 1 000 000.

78. The process as claimed in claim 45, wherein said compound X represents from 0.1% to 95% by weight relative to the total weight of the composition containing it.

79. The process as claimed in claim 45, wherein said compound Y represents from 0.1% to 95% by weight relative to the total weight of the composition containing it.

80. The process as claimed in claim 45, wherein said compounds X and Y are placed in contact in an X/Y mole ratio ranging from 0.05 to 20.

81. A kit for treating keratin material(s) comprising at least two different compositions conditioned separately, the kit comprising at least one filler and/or one wax, at least one compound X, at least one compound Y, with at least one of the compounds X or Y being silicone-based, and at least one catalyst, said compounds X and Y being capable of reacting together via a hydrosilylation reaction in the presence of a catalyst when they are placed in contact with each other and in which compounds X and Y, and the catalyst are not simultaneously present in the same composition, and said filler and/or wax is in an amount sufficient to impart opacity to the filling product resulting from the interaction of compounds X and Y.

82. The kit as claimed in claim 81, comprising at least:
one first composition comprising, in a physiologically acceptable medium, at least one compound X,
one second composition comprising, in a physiologically acceptable medium, at least one compound Y,
with at least one of the first and second compositions also comprising at least one filler and/or one wax, and at least one of said compositions also containing at least one catalyst.

83. The kit as claimed in claim 81, also comprising an anti-aging active agent.

84. The kit as claimed in claim 81, in which at least one of the compositions has, for a shear rate equal to 10⁻³ s⁻¹, a viscosity of greater than 10 000 Pa·s.

85. The kit as claimed in claim 81, wherein both compositions have, for a shear rate equal to 10⁻³ s⁻¹, a viscosity of greater than 10 000 Pa·s.

86. The kit as claimed in claim 81, wherein each composition is conditioned separately in the same conditioning article.

87. The kit as claimed in claim 81, wherein each composition contains at least one filler and/or one wax in an amount sufficient to impart opacity to each of the compositions.

88. The kit as claimed in claim 81, wherein each of the first and second compositions is conditioned in a separate conditioning article.

89. A cosmetic treatment process for improving the surface appearance of the skin which consists in applying to said irregularities a filling product resulting from a condensation reaction, optionally in the presence of a catalyst, between two compounds X and Y, at least one of which is a silicone compound, said compounds being placed in contact with each other optionally in the presence of a catalyst, such that said reaction takes place at least partly on the skin.
